(19) United States
(12) Patent Application Publication
(10) Pub. No.: US 2009/0269362 A1
(43) Pub. Date: Oct. 29, 2009

(54) Method for Controlling Immunodominance

(76) Inventor: Andrea J. Sant, Rochester, NY (US)

Correspondence Address: BLANK ROME LLP, WATERGATE, 600 NEW HAMPSHIRE AVENUE, N.W., WASHINGTON, DC, 20037, US

(21) Appl. No.: 11/996,907

(22) PCT Filed: Jul. 25, 2006
(86) PCT No.: PCT/US2006/028735

§ 371 Date: Dec. 8, 2008

Related U.S. Application Data
(60) Provisional application No. 60/701,971, filed on Jul. 25, 2005.

(51) Int. Cl. A61K 39/00; A61P 35/00; A61P 25/00; A61P 31/12; A61P 31/04; A61P 31/10

(52) U.S. Cl. 424/185.1

(57) ABSTRACT

Methods for controlling immunodominance are described. These methods are carried out by altering the kinetic stability of a complex between a class II Major Histocompatibility Complex (MHC) molecule and the epitope for which immunodominance is to be altered. Alterations that increase the kinetic stability of the epitope: class II MHC complex confer immunodominance upon the epitope. Methods are also described for stimulating an immune response in an organism to a specific epitope by administering to the organism a form of that epitope which has been altered to be immunodominant.

### BACKGROUND OF THE INVENTION

The term immunodominant describes an epitope capable of stimulating an immune response over other potential epitopes contained within a protein or organism. As is well known in the art, antigen presenting cells (APCs) endocytose extracellular proteins and degrade them into peptides with lysosomes. Certain peptides, known in the art as epitopes, are then displayed on the surface of the APCs complexed with Major Histocompatibility Complex (MHC) class II molecules. Only a small subset of epitopes created by APCs actually stimulate a detectable immune response from CD4 helper T-cells. The epitopes that do stimulate a detectable immune response are known as immunodominant. Those epitopes that do not stimulate an immune response are known as cryptic. The focused response to a limited set of peptides within complex proteins reveals a considerable selective pressure on an emerging T cell response.

Previous studies investigating the selectivity of CD4 T cell responses have uncovered several factors that can influence the specificity of T cells including antigen processing and presentation, T cell precursor frequency, and T cell competition (Blum et al. Crit. Rev Immunol 17, 411-417, 1997; Kedl et al., Curr Opin Immunol 15, 120-127, 2003; Manoury et al., Nature Immunology 3, 169-174, 2002; Medd and Chain, Sem. Cell & Dev Bio 11, 203-210, 2000; Sercarz et al, Annu Rev Immunol 11, 729-766, 1993). Historically, processing of native antigen and subsequent presentation by APCs has been thought to be one of the major factors influencing the specificity of T cells. Endosomal proteolytic processing has the potential to either positively or negatively affect-immunogenicity. The assembly of Major Histocompatibility Complex (MHC) class II: peptide complexes is another potential site of regulation. Assembly can be influenced by inter-peptide competition for binding MHC class II molecules' modulation by DM or “epitope capture” by peptides adjacent to the test peptide. The frequency of peptide specific T cells can also influence immunodominance and, in particular, negative selection can delete CD4 T cells specific for immunodominant peptides within self-antigens. Finally, competition between T cells for interaction with APCs is a well-documented phenomenon in the CD8 T cell response and has been proposed to extend to CD4 T cell responses.

The preceding studies suggested that many complex events converge to influence the selective specificity of CD4 T cells during primary immune responses, and the relative contribution of any of these parameters could influence immunogenicity. However, it has also been demonstrated that DM expression singularly enhanced presentation of immunodominant epitopes while antagonizing presentation of cryptic peptides (Nanda and Sant, 2000). This finding suggested that some intrinsic property of the MHC class II: peptide complex itself might be the most important parameter in determining immunodominance.

Current viral vaccine design involves developing vaccines that represent, or are mixtures of, the most prevalent strain or strains of a virus. This is because the cross-reactivity of an immune response directed to one strain of a virus to another strain is unpredictable at best, i.e. a vaccine containing one strain of a virus may of may not produce an immune response to another strain of the same species of virus. This is reflected in the yearly flu vaccine process. Every year, the U.S. Centers for Disease Control (CDC) develops a new flu vaccine “cocktail” based on analysis of flu strains throughout the world and predictions as to which strains will be prevalent. As the CDC readily admits, the effectiveness of the vaccine at preventing flu across the population depends almost entirely on how well the immune response stimulated by the strains in the cocktail recognizes the flu strains that actually develop. Currently, scientists and medical practitioners are unable to control which epitope from a pathogen stimulates an immune response. Often, the immunodominant epitopes of pathogens are derived from regions that undergo high rates of mutation, and hence, are likely to be highly variable from strain to strain of the same species. As such, there is a need in the art for vaccines that stimulate an immune response to epitopes that are highly homologous between various species and stains of pathogens. These vaccines would give much more reliable vaccination, eliminate the need to guess the predominant strains of an outbreak, and would eliminate the need for a “cocktail” vaccination approach.

The ability to control immunodominance has the potential to revolutionize vaccine design. The ability to specifically confer immunodominance on specific epitopes has wide reaching implications for not only vaccination against pathogens, but also for the development of vaccines and treatments for cancer, neurological diseases and other human ailments.

### SUMMARY OF THE INVENTION

It is an object of the present invention to provide a method for controlling immunodominance in an organism. More specifically, it is an object of the present invention to provide a method for conferring immunodominance to a chosen epitope or causing an immunodominant epitope to become cryptic. In one aspect of the method of the present invention, immunodominance is controlled by modifying the kinetic stability of Major Histocompatibility Complex (MHC) class II: peptide complexes. This kinetic stability can be modified by making modifications within the peptide epitope for which one wishes to control immunodominance.

It is a further object of the present invention to provide a method in which mutations can be made within the full-length wild type protein or within other versions of the protein or within the peptide itself.

It is a further object of the present invention to provide a method to modify proteins or peptides within their normal molecular context, followed by immunization of an organism with such modified proteins or peptides, wherein such immunization confers immungenicity onto not only the mutant epitope, but also confers immunogenicity onto the wild type epitope encoded by the organism's genome.

It is a further object of the present invention to create a vaccine that stimulates an immune response to a chosen epitope or set of epitopes.

It is a further object of the present invention is to produce a vaccine that contains an epitope common to as many strains or species of a pathogen as possible.

Other objects, features, and advantages of the present invention will become apparent upon reading the specification and claims.

### DESCRIPTION OF THE DRAWINGS

FIG. 1 shows a comparison between peptide dissociation and peptide competition.

A. The half-life was calculated from the exponential equation fitted to the fluorescence decay curve as a function of the incubation time, and described as the time required to dissociate the 50% of the FITC peptide initially bound to sI-A^{d}.

B. The percentage of inhibition of binding of 1 μM N-terminal FITC-HA[126-138] to sI-A^{d} by the unlabeled competitor peptide was plotted against the concentration of unlabeled inhibitory peptide. Data is represented as a Hill Plot (Hill, J Physiol 40, iv-vii, 1910), and is the average of 2 independent experiments

FIG. 2 shows plots characterizing kinetic stability variants of HA [126-138], LACK [156-173], and HEL [11-25].

A, C and E. Candidate peptide variants were identified by in vitro stimulation of 5×10⁴ specific hybridomas with soluble peptide presented by 4×10⁴ I-A^{d} expressing L cells. IL-2 production by HA TS2 (A), LMR 7.5 (C), and HEL 25 (E) hybridomas in response to 667 nM (A and E) or 300 nM (C) peptide was measured as described in Example 1. Data is representative of 3 independent experiments.

B, D and F. Dissociation of peptides from I-A^{d} was found to fit a single exponential curve with a square correlation coefficient r²>0.99 from which t_{1/2} could be determined. Data is representative of at least 2 independent experiments.

FIG. 3 shows the construction and characterization of hybrid MalE used to incorporate antigenic peptides.

A. DNA encoding antigenic peptides with flanking residues was inserted in-frame into MalE at amino acid 133 via BamHI ligation.

B, C, D. MalE:HA (B), MalE:LACK (C), or MalE:HEL (D) purified from sequenced clones was tested for the ability to activate 5×10⁴ peptide specific hybridomas in vitro using 5×10⁵ BALB/c spleen as APCs. As a measure of T cell stimulation, IL-2 production was assayed by CTLL proliferation using an MTT assay.

FIG. 4 shows that the kinetic stability of peptide:MHC complexes controls in vivo recognition of CD4 T cell epitopes. Groups of 2 mice (A, B, and C, BALB/c; B, B10.D2) were immunized in the footpad with 20 μg/ml (A and B) or 200 μg/ml (C) of the indicated protein emulsified in 50 μl PBS:CFA. The number of IL-2 producing cells at day 10 was determined by 16 h in vitro stimulation of unpurified (A and B) or CD4 purified (C) cells with syngeneic spleen cells and 20 μg/ml (A and B) or 200 μg/ml (C) MalE protein or 5 μM peptide antigen using IL-2 ELISPOT assays. Data represents the mean counts of two separate cell dilutions (A and B, 1×10⁶ and 5×10⁵; C, 5×10⁵ and 2.5×10⁵) of triplicate wells normalized as a percent of the response against the immunizing protein with background spot counts subtracted. Data presented represents the mean of at least 3 independent experiments ±SD.

FIG. 5 shows that the kinetic stability of LACK [156-173] with I-A^{d} controls the immunogenicity within native LACK protein. Groups of 2 BALB/c mice were immunized in the footpad with 200 μg/ml of (A) purified LACK protein or (3) purified LACK:I166A protein emulsified in 50 μL of PBS:CFA. The number of IL-2 producing cells at day 10 was determined by 16 h in vitro stimulation of CD4 purified cells with syngeneic spleen cells and a range of protein or peptide concentrations. Shown are: LACK and LACK 166A protein-200 μg/mL and 8 μg/mL; LACK and LACK:I166A peptide-50 μM and 2 μM; PPD-5 μg/mL and 1 μg/mL. Spots were quantified using triplicate wells of IL-2 ELISPOT assays with background subtracted and are representative of 2 independent experiments.

FIG. 6 shows that increasing the kinetic stability of OVA [327-339] with I-A^{d} enhances immunogenicity. Groups of 2 BALB/c mice were immunized in the footpad with (A) 200 μg/mL OVA protein, (B) 200 μg/mL of the indicated MalE protein, or (D and E) 5 nmol of the indicated peptide emulsified in 50 μL PBS:CFA. The number of IL-2 producing cells at day 10 was determined by 16 h in vitro stimulation of CD4 purified cells with syngeneic spleen cells and (A) 200 μg/mL protein or 12.5 μM peptide, (B) 200 μg/mL protein or 5 μM peptide, or (D and E) the indicated amount of peptide and quantified using IL-2 ELISPOT assays with background subtracted. (A and D) Data represents the mean counts of two separate cell dilutions (2.5×10 and 1.25×10⁵) of triplicate wells normalized as indicated. (C) Dissociation of peptides from I-A^{d} was found to fit a single exponential curve with a square correlation coefficient r²>0.99 from which t_{1/2} could be determined. * indicates no detectable stimulation above background. Data are representative of at least 2 independent experiments.

FIG. 7 describes the identification the binding register of LACK [156-173] and HEL [11-25] to I-A^{d}. (A-C) The LACK [156-173] register was identified using a combination of peptide truncations and variants with substitutions at putative pocket residues. In vitro stimulation of 5×10⁴ LACK-specific hybridomas was measured for candidate peptide truncations and kinetic stability variants presented by 4×10⁴, I-A^{d} expressing L cells at 400 nM (C) or the indicated concentrations (A and B). IL-2 production by 4F7 (A and B) and LMR 7.5 (C) was measured by CTLL proliferation in an MTT assay. * indicates no response above background. (D) To examine the register for HEL [11-25], groups of 2 BALB/c mice were immunized with 5 nmol of HEL[11-25] peptide emulsified in 50 μL PBS:CFA. Draining LN were harvested 10 days later, and the number of IL-2 producing cells was determined by 16 h in vitro stimulation of 1×10⁶ unpurified LN cells with syngeneic spleen presenting 10 μM of the indicated peptides using ELISPOT assay. HEL [11-25] peptide variants spanned four potential registers with pocket interactions in bold type. AMKRHGLDNYRGYSL (R1), AMKRHGLDNYRGYSL (R2), AMKRHGLDNYRGYSL (R3), and AMKRHGLDNYRGYSL (R4). A lack of recognition of peptide variants with substitutions at K13 and H15 by a polyclonal pool of HEL [11-25] reactive cells suggested these residues interacted with the T cell receptor. Lymph node cells specific for HEL [11-25] did recognize peptide variants with substitutions at R14, N19, and G22. Collectively, this suggested the register for HEL [11-25] was AMKRHGLDNYRGYSL. * indicates no spots above background level. Data is representative of 2 independent experiments.

FIG. 8 shows that the dissociation of peptides from I-A^{d}-presenting APCs in vitro reproduces the hierarchy of kinetic stability with I-A^{d}. Dissociation of peptides from 4×10⁴ mitomycin C treated A20 cells was measured by IL-2 production by 5×10⁴ (A and C) DO11.10 hybridomas or (B and D) HA TS2 hybridomas in response to the indicated peptides pulsed on the A20 cells for 30′. For dissociation experiments, concentrations of peptide were selected from the linear portion of the dose response curves (A, 100 μM OVA [327-339] and 10 μM OVA A332I, I334V; B, 0.25 μM HA [126-138], 5 μM HA:T128G, and 5 μM HA:V131A). Stimulation indexes were measured as a percentage of the IL-2 production assayed by OD from time=0 h. As a measure of T cell stimulation, IL-2 production was assayed by CTLL proliferation using an MTT assay. Dissociation of peptides from soluble class II molecules at pH 7.4 for the indicated peptides was: OVA [327-339]-1.3 h; OVA [327-339] A332I, I334V-300 h; HA [126-138]-59 h; HA [126-138] V131A-6 h; HA [126-138] T128G-13 h.

FIG. 9 shows that kinetic stability variants retain cross reactivity as peptide immunogens. Groups of 2 BALB/c (A and C) or B10.D2 (B) mice were immunized with 5 mmol of the indicated peptide emulsified in 50 μL PBS:CFA. Draining LN were harvested 10 days later, and the number of IL-2 producing cells was determined by 16 h in vitro stimulation of unpurified LN cells with syngeneic spleen presenting 5 μM peptide using an ELISPOT assay. Data represents the mean counts of two separate cell dilutions (1×10⁶ and 5×10⁵) of triplicate wells normalized with background spot counts subtracted. Groups of 2 BALB/c mice were immunized with 200 μg/ml of the indicated MalE protein emulsified in 50 μl PBS:CFA (D). Draining LN were harvested 10 days later, and the number of IL-2 producing cells was determined by 16 h in vitro stimulation of CD4+ purified cells with syngeneic spleen presenting 5 μM peptide using an ELISPOT assay. Data represents the mean counts of two separate cell dilutions (5×10⁵ and 2.5×10⁵) of triplicate wells normalized as a percent of the response against the immunizing protein with background spot counts subtracted. Data presented is representative of 4 independent experiments.

### DETAILED DESCRIPTION OF THE INVENTION

The present invention is drawn to methods for controlling the immunodominance of an antigenic epitope. The main characteristics of an immunodominant epitope have been previously unknown in the art. Here, it is set forth in the Examples that immunodominant peptide epitopes form kinetically stable complexes with class II MHC molecules (class II MHC). It is an object of the present invention to control whether or not an epitope is immunodominant by modifying its ability to form an kinetically stable complex with class II MHC.

In one embodiment of the present invention the kinetic stability of class II MHC: peptide complexes is modified through the use of mutations made in the epitope. Using a variety of methods well known in the art, a nucleic acid sequence corresponding to the desired amino acid sequence of the epitope is mutated. This epitope can be mutated in a variety of contexts, preferably in the context of a nucleic acid sequence encoding the full-length wild type protein. Examples of other contexts in which the epitope may be mutated include, but are not limited to, mutations within a nucleic acid sequence encoding an epitope in proteins other than full-length wild type proteins, mutations within a nucleic acid sequence encoding proteins that have already have other, separate mutations affecting catalytic activity or another property, or mutations within a nucleic acid sequence encoding shorter peptide sequences containing the epitope of interest. Another example of a context that falls within the scope of the invention is a mutation in the nucleic acid sequence of an epitope that is present in a nucleic acid sequence in which it is not normally found in nature. A non-limiting example of such a context is put forth the Examples below, where the epitope is present as part of the MalE protein sequence. It should be apparent to one of skill in the art that any context of mutation that results in a peptide epitope produced by an organism's immune system containing the mutation desired for modifying the kinetic stability of the class II MHC: peptide complex falls within the scope of the present invention. It should also be apparent that mutations to the epitope in other contexts still fall within the scope and spirit of the present invention.

In another embodiment of the invention, the epitopes are modified by protein modification. These modifications may be made in the context of the epitope peptide itself, or may be made in the epitope while it is part of a larger peptide or protein. Non-limiting examples of protein modifications include: acetylation—the addition of an acetyl group, usually at the N-terminus of the protein or peptide; alkylation—the addition of an alkyl group (e.g. methyl, ethyl) usually at lysine or arginine residues; biotinylation—acylation of conserved lysine residues with a biotin appendage; glycosylation—the addition of a glycosyl group to either asparagine, hydroxylysine, serine, or threonine, resulting in a glycoprotein; isoprenylation—the addition of an isoprenoid group (e.g. farnesol and geranylgeraniol); lipoylation—attachment of a lipoate functionality; phosphopantetheinylation—the addition of a 4′-phosphopantetheinyl moiety from coenzyme A; phosphorylation—the addition of a phosphate group, usually to serine, tyrosine, threonine or histidine; sulfation—the addition of a sulfate group to a tyrosine; citrullination (deimination)—the conversion of arginine to citrulline; and deamidation—the conversion of glutamine to glutamic acid or asparagine to aspartic acid. The present invention contemplates that modification of the epitope in question may be used to change the kinetic stability of the epitope: class II MHC complex (i.e. to make the complex kinetically more or less stable).

Whether through mutation, protein modification or some other means, the methods of the invention are meant to alter the kinetic stability of the epitope: class II MHC complex. Preferably, modification of the kinetic stability of the complex is effected by changing the shape, structure or charge of one or more of the amino acid side chains of the epitope peptide. Changes in amino acids may be made a specific positions that correspond with specific binding pockets on the class II MHC molecule. The change in the kinetic stability is preferably effected through a change in the disassociation rate (“off” rate) of the epitope: class II MHC complex. Non-limiting examples of changes in amino acid side chains and their affects on the kinetic stability of the complex are shown in the Examples and Table I below. Further examples of changes in the amino acid side chains of the epitope peptide can be found in Lazarski et al., Immunity, 23, 29-40, 2005; Sant et al., Immunological Reviews, 207, 261-278, 2005 and Chaves et al., Biochemistry, 45, 6426-6433, 2006, which are hereby incorporated by reference herein.

In one embodiment of the present invention the mutated epitope is administered—in whatever context it may be present—to the organism to be immunized. In a preferred embodiment, the peptide epitope with the desired mutation or mutations is administered to the organism in the context of a purified protein that is injected into the organism. Another method of administration within the scope of the present invention includes, but is not limited to, delivery of the mutated epitope by administering a nucleic acid sequence encoding the mutated epitope to an organism, for example through the use of viral vectors, such as a herpes simplex virus amplicon. Additionally, one may derive a modified version of the pathogen of interest (for example Influenza virus) that contains the modified peptide epitope allowing use of the modified intact organism as a vaccine to drive the focus of the immune response towards the desired targeted epitope. It should be apparent to one skilled in the art that any method of administering a nucleic acid sequence or amino acid sequence to a patient to stimulate an immune response falls within the scope of the present invention.

In one embodiment of the present invention, a vaccine is produced that is specifically targeted to an epitope or set of epitopes. In a preferred embodiment, the targeted epitope or set of epitopes are present in more than one species or stain of pathogen. As a non-limiting example, through the use of genome sequences well known in the art, a vaccine could be designed containing an epitope whose nucleic acid sequence is present in the genome of many different pathogen species, such as many different species of a virus. Administration of this vaccine would then stimulate an immune response to all of the pathogens containing the chosen epitope. Examples of pathogens for which vaccines could be developed by the present invention include, but are not limited to, viruses such as influenza virus, rhinoviruses, coronaviruses, echoviruses, paramyxoviruses, poxviruses, coxsackieviruses, Human Immunodeficiency Virus (HIV), avian influenza virus, Ebola virus, hepatitis viruses, herpes viruses, papillomavirus, borna virus, yellow fever virus and dengue virus; bacteria such anthrax, streptococcus and staphylococcus; and fungal pathogens. It will be apparent to those of skill in the art that the present invention could also be used to develop vaccines containing epitopes capable of stimulating an immune response to treat or prevent cancer, neurological disease, or other ailments. Such vaccines may work by stimulating an immune response to a specific form of a protein or other factor that is involved in the pathogenic process of said cancer or neurological disease.

The detailed description of the invention and the examples below are meant to set forth certain embodiments of the invention. It will be apparent to one of skill in the art that there are other embodiments not set forth here that still fall within the scope and spirit of the present invention.

### EXAMPLES

### Example 1

### Materials and Methods Used

### Antibodies and Peptides

Purified rat anti-mouse IL-2 (JES6-1A12) antibodies and biotinylated rat anti-mouse IL-2 (JES6-5H4) antibodies were obtained from BD PharMingen. Synthetic peptides were obtained either from commercial sources, or were the generous gifts of C. Beeson (Medical University of South Carolina), N. Glaichenhaus (University of Nice), and D. Fowell (University of Rochester).

### Purification of Soluble I-A^{d} Proteins

A chimeric soluble I-A^{d} protein (sI-A^{d}), with a small segment of the carboxyterminal domains of I-A replaced with I-E sequences was used for peptide binding studies. It has been shown that the modifications improve dimer stability but do not affect peptide-binding characteristics of class II molecules (Chaves et al., J. Immunological Methods 300, 74-92, 2005). Transfectants expressing the PI-linked class II molecules used as a source of class II, which was obtained from detergent lysates by antibody affinity chromatography as described (Chaves et al., J. Immunological Methods 300, 74-92, 2005).

### Dissociation Experiments

sI-A^{d} (50 nM final concentration) was mixed with FITC peptide (5 μM final concentration) in McIlvaines buffer pH 5.3 (0.2 M citric acid, 0.5 M Na₂HPO₄), 0.2 mM n-dodec, 0.025% NaN₃ in the presence of protease inhibitors for 1-16 h at 37° C. sI-A^{d}-FITC-peptide complexes were separated from free FITC-peptide by passage over a Micro Bio-Spin 30 column and the complexes were incubated at 37° C. and pH 5.3 for increasing lengths of time in the presence of 5 μM unlabeled Eα [52-68] peptide to avoid re-binding of the fluorescinated peptide. At each time point, a sample of the dissociation mixture was injected into a LC-10AT HPLC(SHIMADZU Corporation) equipped with a Bio-Sep-SEC-S 3000 column 300×7.8 mm (Phenomenex Inc) connected to an in-line fluorescence detector (RF-10AXL fluorescence detector (SHIMADZU Corporation) as described (Chaves et al., J. Immunological Methods 300, 74-92, 2005).

### Competition Experiments

Soluble I-A^{d} (20 nM) and 1 μM FITC-HA[126-138] were used for competition assays, which is in the titratable range of peptide and class II molecules. Unlabeled competitor peptides at a final concentrations ranging between 0-500 μM were mixed, and after 16-20 h of incubation at 37° C., the sI-A^{d}-FITC-HA[126-138] complex was separated from free peptide and quantified as described above. Inhibition by competitor peptides was calculated using the Hill equation ((Hill, J Physiol 40, iv-vii, 1910), with the IC₅₀ value the concentration of unlabeled competitor peptide required to achieve 50% inhibition of the labeled peptide binding to class II molecules.

### T Cell Hybridoma Assays

The LACK specific hybridoma (4F7) and HA specific hybridoma (TS2) were created by fusion of peptide activated LN cells from the ABLE mouse (Reiner et al., Science 259, 1457-1460, 1998) (4F7) or HNT-TCR mouse (Scott et al., Immunity 1, 73-83, 1994) (TS2) with BW5147 lymphoma cells. T cell assays were performed as previously described in overnight cultures (Peterson and Sant, J Immunol 161, 2961-2967, 1998) with peptide or protein at the specified dose in a flat bottom 96 well dish. IL-2 produced by the T cells was quantified using CTL.L and MTT assays as previously described (Peterson and Sant, J Immunol 161, 2961-2967, 1998).

### Immunizations

BALB/c or B10.D2 mice were immunized in the footpad with 50 μl of 20 μg/ml MalE protein or 5 mmole of peptide emulsified in CFA (Sigma-Aldrich). Ten days later, cells were isolated from draining popliteal lymph nodes. IL-2 production by the unpurified lymph node cells was measured by ELISPOT assay as described previously (Wang and Mosmann, J Exp Med 194, 1069-1080, 2001), using DMEM media with 10% fetal calf serum (Katz et al., J Exp Med 184, 1747-1753, 1996) instead of RPMI, and triplicate wells for each conditions. Quantification of IL-2 producing cells was accomplished with an Immunospot reader series 2A using Immunospot software version 2.0 (Cellular Technologies Ltd).

### MalE Protein Purification

PAGE-purified synthetic oligonucleotides encoding the desired peptide were obtained from IDT DNA technologies and resuspended in 10 mM Tris/1 mM EDTA at a concentration of 100 μM. Annealed double-stranded DNA was ligated into the MalE133 vector and sequenced clones were transformed into MalE (−/−) ER2507 E. coli. MalE protein was prepared as described (Martineau et al., Gene 118, 151, 1992) with some modifications.

### LACK Protein Synthesis

LACK cDNA expression vector (Mougneau et al., Science 268, 563-566, 1995), was mutated at position 166 via Quikchange site directed mutagenesis (Stratagene) and confirmed by sequence analysis. O/N cultures of BL21(DE3λ) bacteria (Novagen) transfected with LACK or LACK:I166A were inoculated into 500 mL of LB with ampicillin and chloramphenicol and grown at 37° C. until an OD₆₀₀ of 0.5 was reached. 0.25 mL of 1 M IPTG was added to induce protein expression and bacteria were grown for another 3 b at 37° C., and subsequently harvested by centrifugation at 5000×g for 15 min 4° C. Pellets were resuspended in 100 mL of 10 mM Imidazole, 50 mM NaHPO₄, 300 mM NaCl pH 8 and sonicated for 1 min. Supernatants were pelleted by centrifugation at 26,000×g for 25 min 4° C. Protein was purified from supernatants via Ni-NTA affinity column and assayed for quantity and purity via SDS-PAGE analysis.

### Example 2

### Kinetic Stability Correlates with Immunodominance

A set of previously identified cryptic and immunodominant epitopes was assembled and characterized for their relative affinity for class II molecules. I-Ad restricted epitopes from divergent origins were utilized, including sperm whale myoglobin (SWM), hen-egg lysozyme (HEL), chicken ovalburnin (OVA), and L. major (LACK) (Mougneau et al., Science 268, 563-566, 1995; Sercarz et al., Annu Rev Immunol 11, 729-766, 1993). The diversity of these epitopes with regard to processing and structure provided the opportunity to isolate a biochemical characteristic that determined in vivo immunodominance. The potential of both peptide competition and peptide dissociation assays was evaluated to distinguish these epitopes. Both assays have been used to determine the relative strength of class II:peptide interactions (Kasson et al., Biochemistry 39, 1048-1058, 2000; McFarland et al., J Immunol 163, 3567-3571, 1999) (Sette et al., J Immunol 142, 35-40, 1989). Peptide competition assays judge the ability of the test peptide to inhibit formation of complexes between a labeled standardized peptide with class II molecules, while dissociation assays directly measure the kinetic stability of interaction between the test peptide and class II molecules after the complexes have been assembled.

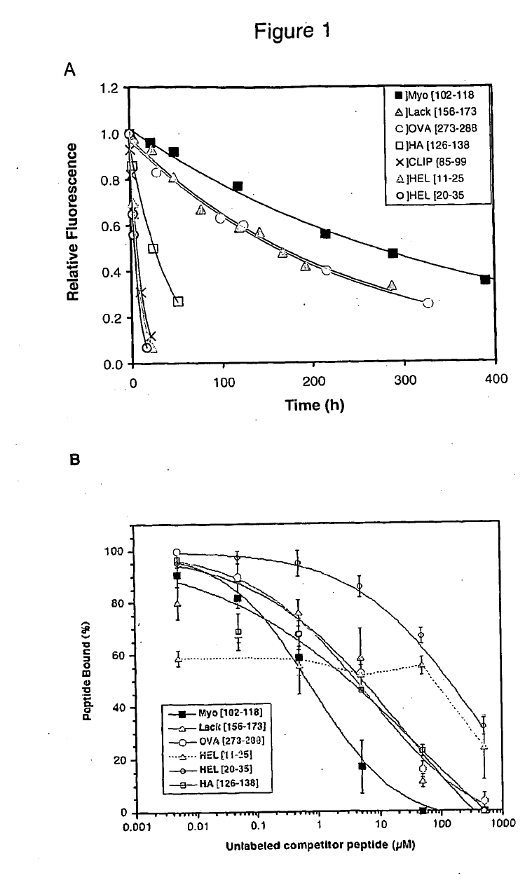

To perform binding studies with I-A^{d}, soluble class II molecules were produced and purified (Chaves et al., J. Immunological Methods 300, 74-92, 2005). For dissociation assays, the half-life of pre-loaded class II I-A^{d} peptide complexes bound with fluorescently-labeled peptide was monitored in vitro at endosomal pH 5.3 and 37° C. Dissociation was found to fit a single exponential curve with a square correlation coefficient r²>0.99 from which the t_{1/2} could be determined. Strikingly, the dissociation curves from FIG. 1A and the t_{1/2} values (shown in Table 1) revealed that immunodominant and cryptic epitopes segregated at opposite ends of a very broad kinetic stability spectrum. The immunodominant epitopes LACK [156-173] (t_{1/2}=170 h), SWM [102-118] (t_{1/2}=260 h), and OVA [273-288] (t_{1/2}=160 h) all displayed long half lives of 150 h or greater. In contrast, the cryptic epitopes HEL [11-25] (t_{1/2}=6 h) and HEL [20-35] (t_{1/2}=4 h) both displayed very short half-lives. Collectively, these results suggest that kinetic stability in class II:peptide complexes may be a critical characteristic that determines immunogenicity of a peptide during an immune response.

In contrast to the results involving kinetic stability measurements, cryptic and immunodominant peptides displayed no consistent groupings when assayed by competition (FIG. 1B). For example, the classically defined cryptic HEL [11-25] peptide competed well with HA [126-138] for binding to I-A^{d}, while the similarly cryptic HEL [20-35] peptide competed poorly with HA [126-138] for binding to I-A^{d}, with a predicted IC₅₀>100 μM. Also, HA [126-138], LACK [156-173] and OVA [273-288] clustered together, even though their potency in immunodominance assays are clearly distinct. Collectively, the studies with these unrelated antigens revealed that under the conditions used in these experiments, dissociation rates of peptides from class II molecules dramatically segregated immunodominant from cryptic peptides. Therefore this parameter was used for the remainder of the experiments. In analyses of many peptides whose dissociation rates differ by seven orders of magnitude, it has been reported (Kasson et al., Biochemistry 39, 1048-1058, 2000) that the association rates among these peptides were essentially the same. This indicates that measurements of dissociation rates of peptides from class II molecules provide a good measure of relative affinities. Therefore, in general, values for “affinity” and “dissociation rates” will agree with each other. However, the terms “kinetic stability” and “off-rates” are used in this specification because that is the parameter that varied and measured in these studies.

TABLE 1
Kinetic stability of wild type and variant peptide
epitopes in association with I-A^{d}.

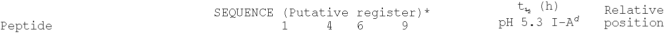

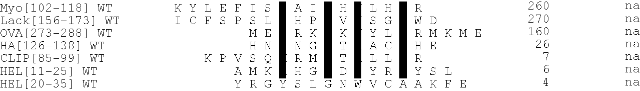

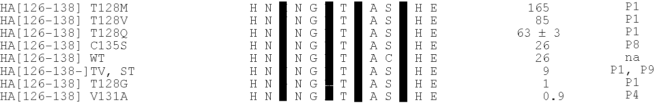

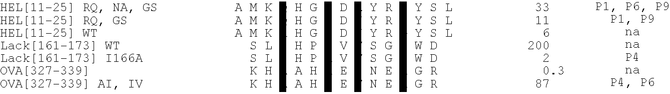

The half-life was calculated from the exponential equation fitted to the fluorescence decay curve as a function of the incubation time, and described as the time required to dissociate the 50% of the FITC peptide initially bound to sI-A^{d}.
*indicates the binding register for HEL [20-35] is unknown.
Cysteine 135 in HA ]126-138] is necessary for T cell activation, but does not affect stability in association with I-A^{d.}
Lys 327 in OVA [327-339] was substituted for Val to eliminate alternate register binding.
Data is representative of at least 2 independent experiments.

### Example 3

### Derivation of Peptide Kinetic Stability Variants

It was desired to extend the correlative findings between class II:peptide half-lives and immunogenicity to test whether a causative relationship between these two parameters could be shown. To address this, peptide variants that possessed increased or decreased kinetic stability with I-A^{d} were sought and then investigated whether changing the kinetic stability of a given class II:peptide complex caused a corresponding change in its immunogenicity in vivo.

In order to arrive at generalizable conclusions, three unrelated peptides were chosen: the influenza HA [126-138] peptide, the LACK [156-173] peptide from L. major, and hen-egg lysozyme (HEL) [11-25], each of which offered unique biological or biochemical properties. The HA [126-138] peptide was chosen because the crystal structure of HA [126-138]: I-A^{d} has been solved (Scott et al., 1998), providing the register for the peptide bound to I-Ad. A second advantage of the HA [126-138] peptide is its intermediate dissociation rate (t_{1/2}=26 h), which provided an opportunity to investigate the biological properties of both higher and lower stability variants with I-A^{d}. The LACK [156-173] peptide from L. major was selected because it is a prototypical immunodominant epitope from a model protozoan infection (Mougneau et al., Science 268, 563-566, 1995; Reiner et al., Science 259, 1457-1460, 1993). This epitope has been found to have a high number of T cell precursors (Milon et al., J Immunol 136, 1467-1471, 1986; Stetson et al., Immunity 17, 191-200, 2002), a property that offered the opportunity to determine whether reducing kinetic stability of class II:peptide complexes would be sufficient to overcome precursor frequency advantages. The HEL [11-25] peptide is a prototypic cryptic peptide (Moudgil et al., 1997) and thus provided an opportunity to reverse apparent sequestration of a peptide from an immune response solely by stabilizing the interaction of the peptide with class II molecules.

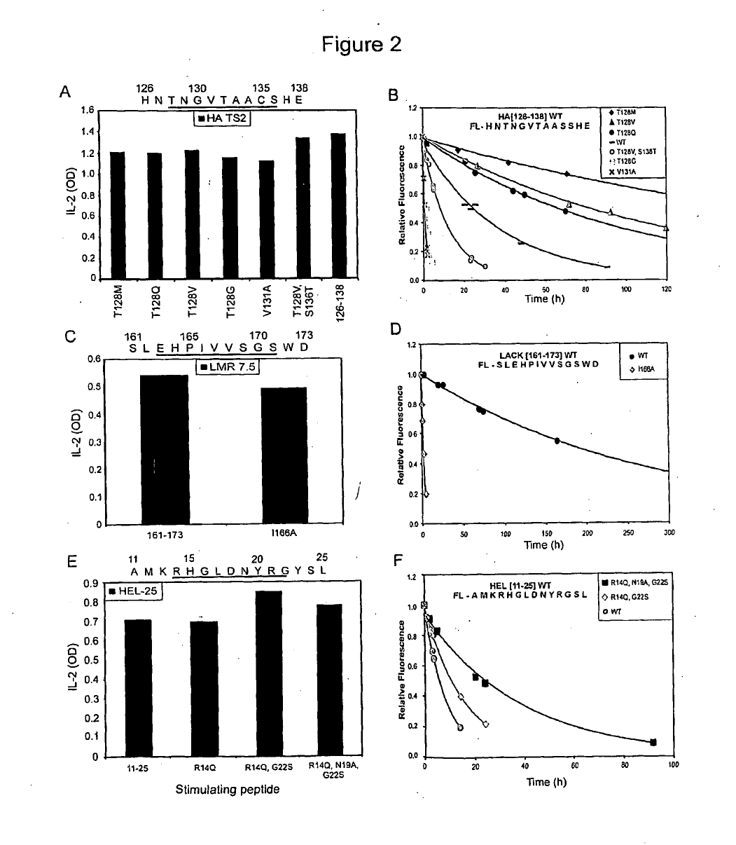

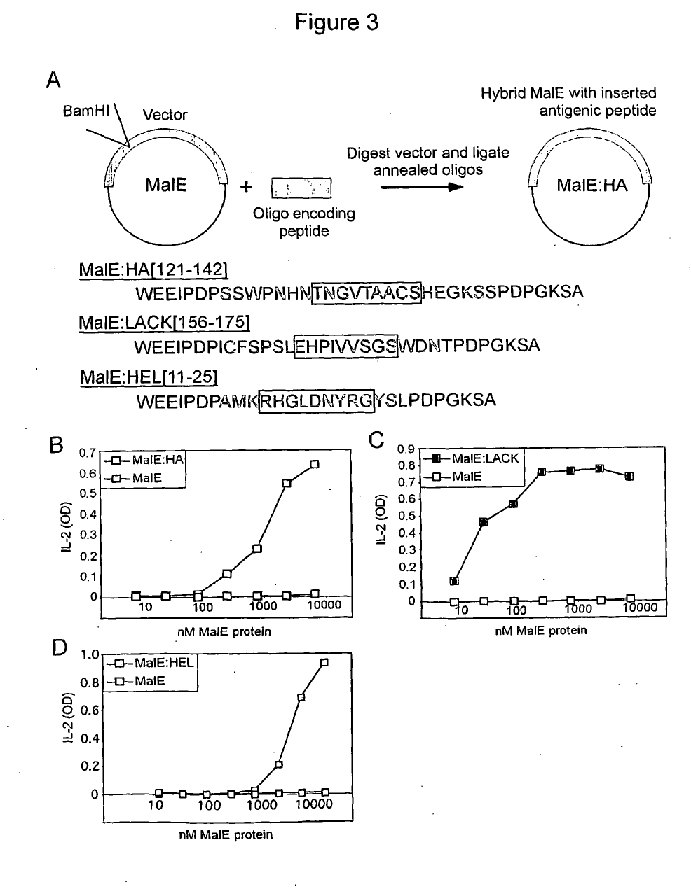

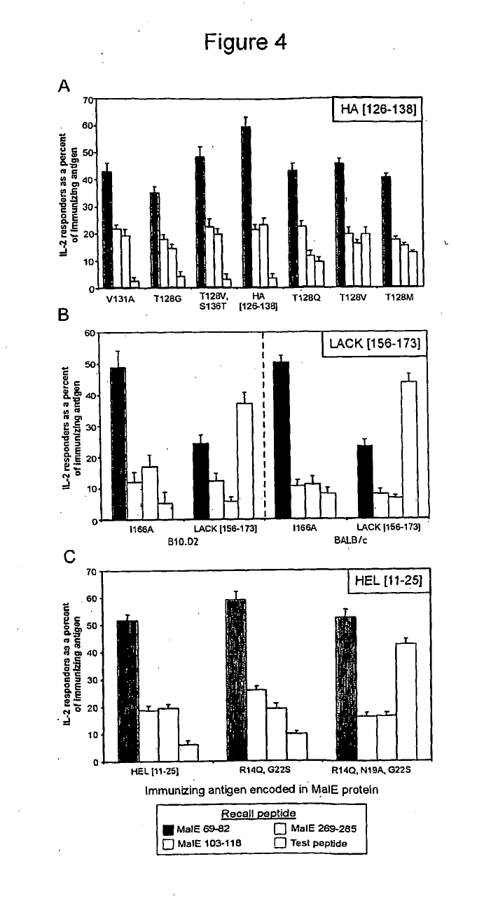

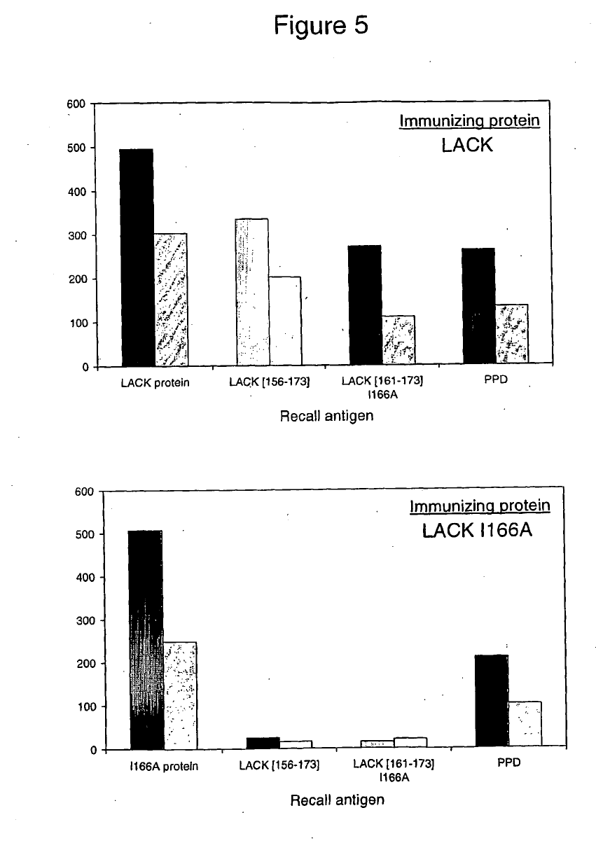

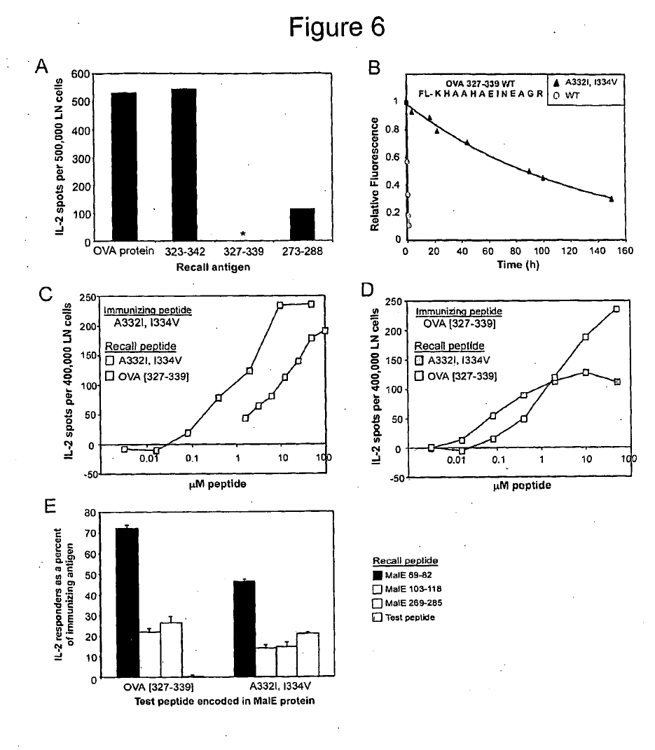

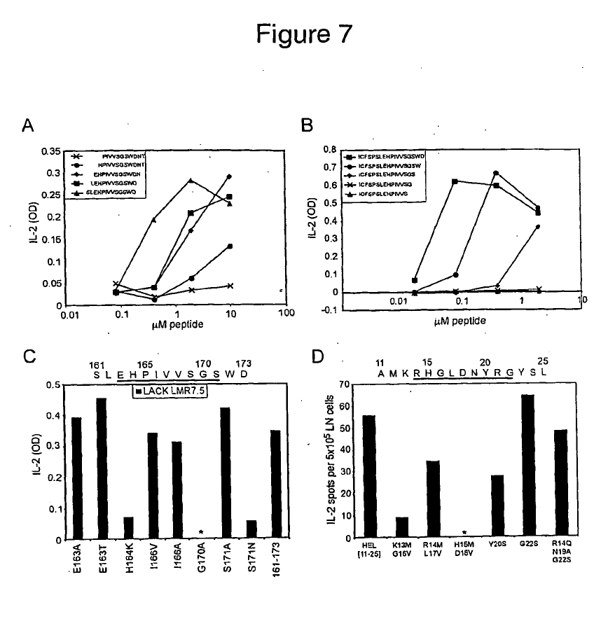

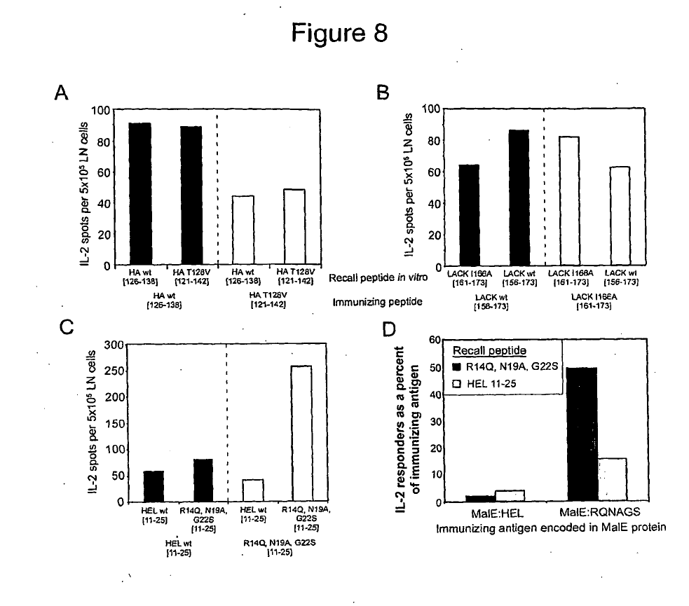

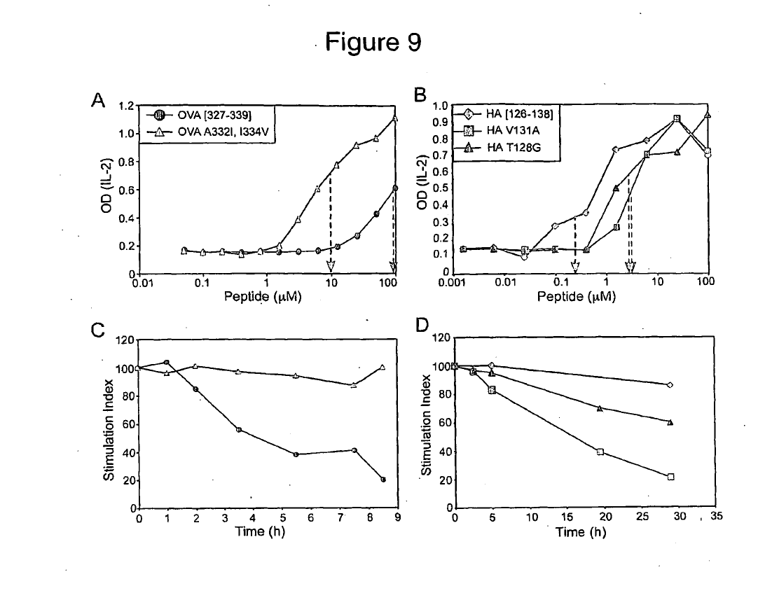

Initial experiments tested the ability of candidate variant peptides to maintain T cell stimulatory capacity when tested with antigen specific T cell hybridomas. Peptide variants that passed this initial screen were evaluated for dissociation kinetics. HA [126-138] variants included substitutions at P1, P4, or P9 pocket residues. T cells responded to most HA [126-138] variant peptides in vitro when presented by I-Ad expressing cells (FIG. 2A). Compared to WT HA [126-138] that displayed a kinetic stability of approximately 26 h with I-A^{d}, three P1 mutants displayed increased stability with I-A^{d} molecules ranging from 63 to 165 h (Table 1 and FIG. 2B). Lower stability variants showed half lives of 1 and 0.9 h respectively (Table 1 and FIG. 2B). The variants summarized in Table 1 provided a range of kinetic stabilities to study and also highlighted the importance of single amino acid interactions with the class II pockets upon the overall stability of the class II:peptide complex.

While the successful crystallization of I-A^{d}:HA [126-138] facilitated design of variant peptides, the core binding sequence of LACK [156-173] was not known. Use of truncated peptides (FIGS. 7A and 7B) indicated the binding core to be residues [163-171] and suggested the following amino acid register from P1 to P9: EHPIVVSGS. This register fits with some of the known binding preferences for I-A^{d}, particularly Ile at P4 and Val at P6. To derive lower kinetic stability variants for LACK, Ala was substituted for Ile at P4 (I166A). This variant sustained T cell stimulation to WT LACK [161-173] (FIG. 2C) and displayed a half-life of only 2 h, nearly 100-fold less than WT LACK (FIG. 2D). The changes in stability observed with I166A demonstrated this residue interacted with I-A^{d} pockets as hypothesized, and verified this register for LACK [156-173]:I-A^{d}.

Determination of the register of the HEL [11-25] peptide presented us with unique challenges. Because of its extremely weak interaction with I-A^{d}, this peptide was not expected to possess even the poorly defined “motif” for I-A^{d} (Sette et al., J Immunol 142, 35-40, 1988). As shown, functional studies were used to determine its binding register with I-A^{d}. These analyses suggested the likely register for the HEL [11-25] peptide with I-A^{d} was AMKRHGLDNYRGYSL, with the bold residues indicating P1, P4, P6 and P9. Higher stability variants of HEL [11-25] displayed half-lives of 11 or 35 h, respectively (FIG. 2F).

### Example 4

### Implementation of a Peptide Shuttle Vector for CD4 T Cell Epitopes

To study the relationship between peptide off-rates and immunogenicity, a protein shuttle vector that could accept heterologous peptide inserts was required. To prevent self-reactive T cells from interfering with responses to inserted epitopes, a vector was used that had no murine homolog. The protein vector chosen, MalE, encodes a subunit of the E. coli maltose binding protein and can accept inserts of greater than twenty amino acids (Martineau et al., Gene 118, 151, 1992). Using the same protein vector for all of the test peptides has the advantage of providing the same set of competing peptides, thus controlling for T cell competition events and allowing responses to be tracked to these MalE peptides in all the immunization studies. To take advantage of this, the immunodominant epitopes within MalE were characterized and it was found that in BALB/c mice, MalE [69-82] was consistently dominant, while MalE [103-118] and MalE [269-285] were subdominant.

In an effort to equalize three-dimensional context and protease sensitivity among the variant or WT peptides, a single insertion site for the peptides was chosen. Also, because insertions that perturb structure diminish affinity to maltose, the purification strategy chosen was based on the functional association of peptide-inserted MalE with cross-linked amylose (Martineau et al., Gene 118, 151, 1992). DNA encoding each peptide of interest was inserted into amino acid 133 flanked by five to seven carboxyl-terminal and amino-terminal residues (FIG. 3A) of the native peptide to preserve potential TCR contacts (Arnold et al., J Immunol 169, 739-749, 2002). With insertion of the HA [126-138], LACK [156-173], and HEL [11-15] peptide epitopes into MalE, the respective T cell hybridomas gained reactivity (FIG. 3B-D), indicating that the inserted peptides were liberated during processing of the protein.

### Example 5

### Immunogenicity of Peptide Variants In Vivo

To examine the immunogenicity of the heterologous test peptide variants within MalE, IL-2 ELISPOT assays were used to quantify the number of CD4 T cells specifically responding to peptides ex vivo. To compare data collected from independent experiments, spot counts were normalized for all the tested peptides relative to the total number of T cells that responded in vitro to the original MalE:insert protein used for immunization. Data shown represent the average of at least three independent experiments. HA [126-138] was found to be cryptic in BALB/c mice when inserted into MalE. Very few T cells specific for HA [126-138] could be detected in immunized mice. The occasional single spot above background corresponded less than 1 in 500,000 lymph node cells. Thus, a kinetic stability of 26 h is insufficient for recognition of HA [126-138]. It was next investigated whether variants of HA [126-138] which displayed increased kinetic stability could overcome the crypticity of WT HA [126-138]. BALB/c mice were immunized with the high stability variant HA T128V (t_{1/2}=86 h) encoded in MalE (MalE:T128V). Strikingly, this assay revealed that the higher stability HA peptide successfully recruited T cells in vivo. Approximately 20% of the specific response was dedicated to the HA variant peptide, similar to the magnitude of MalE [103-118] and MalE [269-285] specific responses (FIG. 4A), demonstrating a half-life of 86 h is sufficient for recognition of HA [126-138] in vivo. It was also found that the two other high stability variants of HA gained immunogenicity. The number of CD4 T cells responding to HA T128M (t_{1/2}=165 h) was also equivalent to the two subdominant backbone MalE peptides previously identified. When inserted into MalE, the HA:T128Q variant that displayed a kinetic stability of 63 h was more immunogenic than the WT peptide, although not as potent as the higher stability variant. In contrast, when low stability variants of HA were inserted into MalE and tested for immunogenicity, these peptides were not immunogenic. Responses to HA V131A (P4) and T128G (P1), both of which displayed quite low kinetic stability interactions with I-A^{d} with t_{1/2} of 3 and 1 h, respectively (FIG. 4B), represented less than 4% of the overall response (FIG. 4A). Finally, the pattern of immunodominance gained with the HA T128V peptide was able to be reversed. A new variant of T128V described above was designed to reduce its stability with I-A^{d} molecules. By substituting the P9 residue Ser in HA with the closely related Thr residue, the kinetic stability of HA T128V was reduced from 85 h to 9 h (FIG. 2B). As shown in FIG. 4A, the T128V, S136T variant of HA became cryptic in the CD4 T cell response when inserted into MalE. These data further support the idea that the immunogenicity of a peptide can be up or down regulated solely by changing its kinetic stability with class II molecules.

Recognition of the LACK [156-173] peptide epitope inserted within MalE (MalE:LACK) as a protein immunogen for H-2^{d} mice was then investigated. The results of this experiment demonstrated LACK [156-173]-specific T cells dominated the in vivo response, in fact surpassing the response to the MalE epitopes (FIG. 4B). Over 1 in 16,000 lymph node cells recognized LACK [156-173], which corresponded to an average of 37% of the total response to MalE:LACK protein in B10.D2 mice, and 44% in BALB/c mice. These results show that the kinetically stable LACK [156-173] epitope is immunodominant within MalE. To determine whether reducing the kinetic stability of LACK with I-A^{d} would be sufficient to extinguish-its immunodominance, the I166A variant of LACK [156-173] (t_{1/2}=2 h) was tested (FIG. 2D). The results of this experiment (FIG. 4B) showed a lack of priming toward the low stability LACK:I166A epitope when inserted within MalE. T cells from MalE:I166A immunized mice did not respond detectably to either the I166A variant peptide or WT LACK [156-173] peptide. The number of lymph node cells-specifically responding to LACK dropped precipitously, corresponding to fewer than 1 in 500,000 lymph node cells. Responses against the MalE protein and backbone MalE peptides were clearly evident, indicating the MalE:I166A variant protein was effectively processed by APCs and that other MalE peptides within it were successfully presented. These results suggest that by reducing the kinetic stability of the binding of LACK to the I-A^{d} molecule, the in vivo response to a normally immunodominant peptide has been successfully eliminated.

To address whether the modulation of immunodominance using the MalE shuttle vector is unique to this expression system, the LACK [156-173] epitope was mutated in its normal molecular context. Recombinant LACK protein containing the WT peptide sequence or with a mutation at residue I166A described above used to immunize BALB/c mice. T cells from the draining lymph node were tested for reactivity with the intact LACK protein, the WT LACK peptide, the I166A variant peptide, or PPD as an immunization control (FIG. 5). The results of this experiment confirmed the results obtained with the MalE peptide shuttle protein. The strongly immunodominant LACK [156-173] peptide can be rendered cryptic by simply reducing the stability of its interaction with I-A^{d}. These results also show that the loss in immunodominance is not due to loss of T cell reactivity, because the LACK:I166A variant peptide effectively stimulates the T cells that were primed against the WT protein.

To extend these studies to a third antigen, the cryptic HEL peptide (Moudgil et al., 1997) and its variants were analyzed for immunogenicity. When incorporated into the MalE protein vector, the failure in immunogenicity of the HEL [11-25] peptide persisted (FIG. 4C). A higher stability variant (t_{1/2}=11 h; FIG. 2F) with changes at P1 and P9 (HEL R14Q, G22S) was incorporated into MalE and tested in vivo, and it remained non-immunogenic. However, when an HEL peptide variant with changes at P1, P6, and P9 (R14Q, N19A, G22S; t_{1/2}=35 h; FIG. 2F) was inserted into MalE, a significant gain in immunogenicity was observed, suggesting a peptide ordinarily sequestered from an immune response can become immunogenic by enhancing the stability of its interaction with class II molecules.

### Example 6

### Kinetic Stability Variants Retain Cross Reactivity In Vivo

The possibility existed that novel TCR contact profiles had been created with the designed kinetic stability variants which either enhanced or abrogated T cell recognition compared to WT peptides. To evaluate this issue comprehensively, mice were immunized with WT or variant peptides and T cells were tested for recognition of both WT and variant peptide (FIG. 8). No change in the number of lymph node cells which recognized the WT HA [126-138] or T128V variant peptide within each immunization condition (FIG. 8A) was observed. Additionally, when tested for cross reactivity, no significant difference in the number of lymph node cells recognizing the WT or low stability LACK [156-173] variants (FIG. 8B) was found. The vigorous responses to both HA [126-138] and LACK:I166A [156-173] as peptide immunogens also demonstrate that a lack of T cell precursors is not the explanation for the observed crypticity of the epitopes within MalE. The HEL variants were not as straightforward in their phenotype. When mice were immunized with the HEL:R14Q, N19A, G22S peptide, a significant portion of the T cells elicited were non-reactive with the WT HEL peptide (FIG. 8C), suggesting this peptide variant offers additional epitopic residues and can thus recruit T cells with additional specificities. To address the relative contribution of epitopic changes versus kinetic stability in the immunogenicity of the HEL peptides, mice were immunized with MalE containing either the WT HEL or R14Q, N19A, G22S peptide inserts. Although some of the gain in reactivity is attributable to recruitment of T cells of new specificity, a significant portion is due solely to the increased stability of the R14Q, N19A, G22S peptide complexed with I-A^{d} (FIG. 8D).

### Example 7

### The Normally Cryptic OVA [327-339] Epitope Recognized by the 3DO11.10 TCR can be Made Immunodominant

To provide another example of a cryptic epitope that can gain immunodominance by simply changing its kinetic stability with I-A^{d}, one of the peptide registers contained within the prototypic I-A^{d}-restricted peptide OVA [323-339] (Jenkins et al., Annu Rev Immunol 19, 23-45, 2001; Sette et al., J Immunol 142, 35-40, 1988) was studied. Several studies (McFarland et al., Biochemistry 38, 16663-16670, 1999; Robertson et al., J Immunol 164, 4706-4712, 2000) have shown that this long peptide contains several alternative registers, including the most amino terminal segment that was co-crystallized with I-A^{d} (Scott et al., Immunity 1, 73-83, 1998). Evavold (Robertson et al., J Immunol 164, 4706-4712, 2000) and Kappler (personal communication) showed the register recognized by the 3DO11.10 T cell is the most carboxyterminal segment [327-339] amino acid with residue 329 constituting the P1 position, which was confirmed through the use of the truncated peptide 327-339. When the stability of this peptide with I-A^{d} was measured (FIG. 6A), it was found, surprisingly, that it displayed a very low kinetic stability of 0.3 h. To test whether this low stability peptide was cryptic, mice were immunized with the OVA protein and tested for T cells that recognize OVA [327-339]. Less than 1% of T cells raised against OVA recognize OVA [327-339] (FIG. 6B), indicating that it is cryptic. A variant with substitutions at P4 and P6 (A332I, I334V), anticipated to improve binding to I-A^{d}, was recognized by the 3DO11.10 T cell hybridoma and showed a greatly enhanced stability with I-A^{d}, with a t_{1/2} greater than 80 h (FIG. 6B). Additionally, T cells raised against the P4/P6 variant cross-reacted back onto the WT OVA peptide (FIGS. 6C and D). To evaluate dominance of the higher stability peptide, both the WT and variant OVA peptides were incorporated into MalE and tested for immunogenicity in BALB/c mice (FIG. 6E). These studies revealed that improved P4 and P6 pocket interactions converted the cryptic OVA peptide into an immunodominant peptide. Importantly, the T cells elicited against the modified peptide cross react with the wild type peptide, indicating that use of peptides with modified interactions with MHC molecules allow expansion of T cells that can detect the original unmodified peptide in association with MHC class II molecules. This finding has important implications for vaccine design and indicate that the use of modified immunogens can lead to priming of T cells that can cross react with the epitopes generated by the original, unmodified pathogen. Collectively, these results suggest, unlike the situation with the HEL [11-25] peptide, the gain in immunogenicity of the OVA peptide can be accounted for by its more stable interaction with the class II presenting molecule.

What is claimed is:

1. A method for making a vaccine which stimulates an immune response to a chosen epitope, said method comprising,
modifying said epitope in a manner that increases the kinetic stability of a complex of a class II MHC molecule and said modified epitope, and
administering said modified epitope to an organism in a manner that causes stimulation of the immune response to said epitope.

2. The method of claim 1, wherein the modified epitope is modified by mutation of its nucleic acid sequence.

3. The method of claim 1, wherein the modified epitope is modified by modification of its amino acid sequence.

4. The method of claim 1, wherein the method of administering the modified epitope is through administration of a purified or partially purified protein containing the modified epitope.

5. The method of claim 1, wherein the administration of the purified or partially purified protein is by injection.

6. The method of claim 1, wherein the method of administering the modified epitope is through the administration of a nucleic acid sequence containing the nucleic acid sequence encoding the modified epitope.

8. The method of claim 1, wherein the method of administering the modified epitope is through the administration of a peptide containing the epitope.

9. The method of claim 1, wherein the immune response stimulated is an immune response to a pathogen.

10. The method of claim 9, wherein the pathogen is a virus.

11. The method of claim 9, wherein the pathogen is a bacteria.

12. The method of claim 9, wherein the pathogen is a fungus.

13. The method of claim 1, wherein the immune response stimulated is an immune response capable of treating cancer.

14. The method of claim 1, wherein the immune response stimulated is an immune response capable of preventing cancer.

15. The method of claim 1, wherein the immune response stimulated is an immune response capable of treating a neurological disease.

16. The method of claim 1, wherein the immune response stimulated is an immune response capable of preventing a neurological disease.

17. A method for controlling the immunodominance of a chosen epitope, said method comprising,
modifying said epitope in a manner that modifies the kinetic stability of a complex of a class II MHC molecule and said modified epitope.

18. The method of claim 17, wherein the modified epitope is modified by mutation of its nucleic acid sequence.

19. The method of claim 17, wherein the modified epitope is modified by modification of its amino acid sequence.

20. The method of claim 17, wherein the modified epitope is modified to become immunodominant.

21. The method of claim 17, wherein the modified epitope is modified to become cryptic.
